SEQUENCE LISTING (24 sequences)

SEQ ID NO: 1 - DNA, 22 bp, Artificial Sequence
  sense primer
ctgagtatgtcgtggagtctac

SEQ ID NO: 2 - DNA, 22 bp, Artificial Sequence
  antisense primer
gttggtggtgcaggatgcattg

SEQ ID NO: 3 - DNA, 20 bp, Artificial Sequence
  sense primer
ccaccacgctcttctgtcta

SEQ ID NO: 4 - DNA, 20 bp, Artificial Sequence
  antisense primer
agggtctgggccatagaact

SEQ ID NO: 5 - DNA, 20 bp, Artificial Sequence
  sense primer
cttcacaagtcggaggctta

SEQ ID NO: 6 - DNA, 20 bp, Artificial Sequence
  antisense primer
ttctgcaagtgcatcatcgt

SEQ ID NO: 7 - DNA, 20 bp, Artificial Sequence
  sense primer
gctgaaagctctccacctca

SEQ ID NO: 8 - DNA, 20 bp, Artificial Sequence
  antisense primer
aggccacaggtattttgtcg

SEQ ID NO: 9 - DNA, 20 bp, Artificial Sequence
  sense primer
cccagaaatcaaggagcatt

SEQ ID NO: 10 - DNA, 20 bp, Artificial Sequence
  antisense primer
ctcttcacctgctccactgc

SEQ ID NO: 11 - DNA, 20 bp, Artificial Sequence
  sense primer
ggcctggaactctgttatcg

SEQ ID NO: 12 - DNA, 20 bp, Artificial Sequence
  antisense primer
gtccctcttggctcttcctc

SEQ ID NO: 13 - DNA, 20 bp, Artificial Sequence
  sense primer
cagcctcaagatcatcagca

SEQ ID NO: 14 - DNA, 20 bp, Artificial Sequence
  antisense primer
ggtcatgagtccttccacga

SEQ ID NO: 15 - DNA, 20 bp, Artificial Sequence
  sense primer
agcccatgttgtagcaaacc

SEQ ID NO: 16 - DNA, 20 bp, Artificial Sequence
  antisense primer
tgaggtacaggccctctgat

SEQ ID NO: 17 - DNA, 22 bp, Artificial Sequence
  sense primer
ccaggagaagattccaaagatg

SEQ ID NO: 18 - DNA, 22 bp, Artificial Sequence
  antisense primer
ggaaggttcaggttgttttctg

SEQ ID NO: 19 - DNA, 20 bp, Artificial Sequence
  sense primer
gtggcaatgaggatgacttg

SEQ ID NO: 20 - DNA, 20 bp, Artificial Sequence
  antisense primer
ggagattcgtagctggatgc

SEQ ID NO: 21 - DNA, 20 bp, Artificial Sequence
  sense primer
tgccttcagcagagtgaaga

SEQ ID NO: 22 - DNA, 20 bp, Artificial Sequence
  antisense primer
ctcagacaaggcttggcaac

SEQ ID NO: 23 - DNA, 21 bp, Artificial Sequence
  sense primer
actgttgtccctcctctccct

SEQ ID NO: 24 - DNA, 20 bp, Artificial Sequence
  antisense primer
gacactgctttggctgcttg

(19) United States
(12) Patent Application Publication
(10) Pub. No.: US 2023/0066156 A1
(43) Pub. Date: Mar. 2, 2023

(54) 6-SUBSTITUTED, 22-CYANO HYODEOXYCHOLANIC ANALOGUES AND USES THEREOF

(71) Applicant: PRECISION BIO-THERAPEUTICS S.R.L., Perugia (IT)

(72) Inventors: Angela Zampella, Napoli (IT); Stefano Fiorucci, Perugia (IT)

(21) Appl. No.: 17/783,336

(22) PCT Filed: Dec. 9, 2020
(86) PCT No.: PCT/IB2020/061695

§ 371 Date: Jun. 8, 2022

(30) Foreign Application Priority Data
Dec. 9, 2019 (IT) 102019000023403

(51) Int. Cl. C07J 41/00

(52) U.S. Cl. CPC C07J 41/0094

(57) ABSTRACT

The present invention relates to compounds of formula (I), to pharmaceutical compositions thereof and to their uses, in particular in the treatment and/or prevention of GPBAR1 mediated diseases.

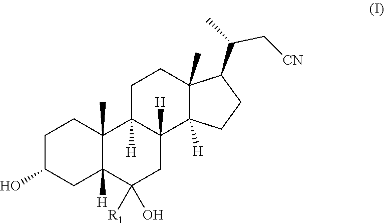

### CROSS-REFERENCE TO RELATED APPLICATIONS

This patent application claims priority from Italian patent application no. 102019000023403 filed on Sep. 12, 2019, the entire disclosure of which is incorporated herein by reference

### TECHNICAL FIELD

The present invention relates to hyodeoxycholic acid derivatives and their use in the treatment and/or prevention of GPBAR1 mediated diseases.

### BACKGROUND ART

Hyodeoxycholic acid belongs to endogenous bile acids (BAs), interacting with at least five types of receptors belonging to the nuclear receptor (NR) super-family: farnesoid X receptor (FXR), identified as the sensor of endogenous bile acids (Makishima et al. Science 1999, 284, 1362. Parchi et al. Science 1999, 284, 1365), constitutive androstane receptor (CAR, identified in Saini et al. Mol. Pharmacol. 2004, 65, 292-300), pregnane X receptor (PXR, identified in Staudinger et al. Proc. Natl. Acad. Sci. U.S.A. 2001, 98, 3369), liver X receptor (LXR, identified in Song et al. Steroids 2000, 65, 423) and vitamin D receptor (VDR, identified in Makishima et al. Science 2002, 296, 1313).

In addition, the secondary bile acids activate G-protein-coupled receptors (GPCR), including GPBAR1 (also known as M-BAR, TGR5, or BG37) (Takeda et al. FEBS Lett. 2002, 520, 97; Kawamata et al. J. Biol. Chem., 2003, 278, 9435).

TGR5/GPBAR1 ligands increase the intracellular concentrations of cAMP with consequent activation of a signalling cascade. GPBAR1 is highly expressed in the liver and in the intestine, but also in the muscles, in the adipose tissue, in the macrophages and in the endothelial cells. In muscles and brown adipose tissue, GPBAR1 increases energy expenditure and oxygen consumption (Watanabe et al. Nature del 2006, 439, 484). In the entero-endocrine L cells, GPBAR1 activation stimulates the secretion of glucagon-like peptide (GLP-1), regulating glucose blood levels, gastrointestinal motility and appetite (Thomas et al. Cell. Metab. 2009, 10, 167).

For all these reasons, the development of GPBAR1 agonists represents an intriguing strategy for the treatment of metabolic disorders like type II diabetes, obesity, inflammatory diseases and some types of cancer.

Hyodeoxycholic acid is generated in human small intestine by a bacterial C-6 hydroxylation of lithocholic acid (LCA). [Eyssen et al. Appl. Environ. Microbiol. 65, 3158-3163 (1999)].

In vitro studies showed that hyodeoxycholic acid (HDCA) is a weak GPBAR1 agonist [Song, C. et al. Steroids 65, 423-427 (2000); Sato, H. et al. J. Med. Chem. 51, 1831-1841 (2008)].

### Disclosure of Invention

The object of the present invention is the identification of new compounds deriving from the hyodeoxycholic acid (HDCA) able to modulate GPBAR1.

Said object is achieved by the present invention, relative to compounds of Formula (I) according to claim 1, their compositions according to claim 4 and to the use thereof according to claims 5 and 6. Preferred embodiments are set out within the dependent claims.

### BRIEF DESCRIPTION OF THE DRAWINGS

The present invention will now be described in detail with reference to the figures of the annexed drawings, in which:

FIG. 1 illustrates the in vitro effects of selected compounds on human monocytes activation measuring relative mRNA expression of pro- and anti-inflammatory genes, IL-1β, IL-6, TNF-α, IL-10;

FIG. 2 illustrates (A) Changes in body weight and (B) Colitis Disease Activity Index (CDAI) of mice during the course of TNBS-induced colitis. (C) H&E staining of colon sections from control mice, mice treated with TNBS, and mice treated with TNBS plus PTB110B (original magnification 10×). (n=5), #p<0.05.

FIG. 3 illustrates relative mRNA expression of pro- and anti-inflammatory genes assessed by Real-Time PCR colon samples: (A) Il-1β, (B) Il-6, (F) Tnf-α, (D) Il-10 and (E) Gpbar1 relative mRNA expression.

FIG. 4 illustrates the relative mRNA expression of pro- and anti-inflammatory genes evaluated in colon samples by real-time PCR: (A) Il-1(3, (B) Il-6, (F) Tnf-α, (D) Il-10, and (E) Gpbar1 mRNA expression.

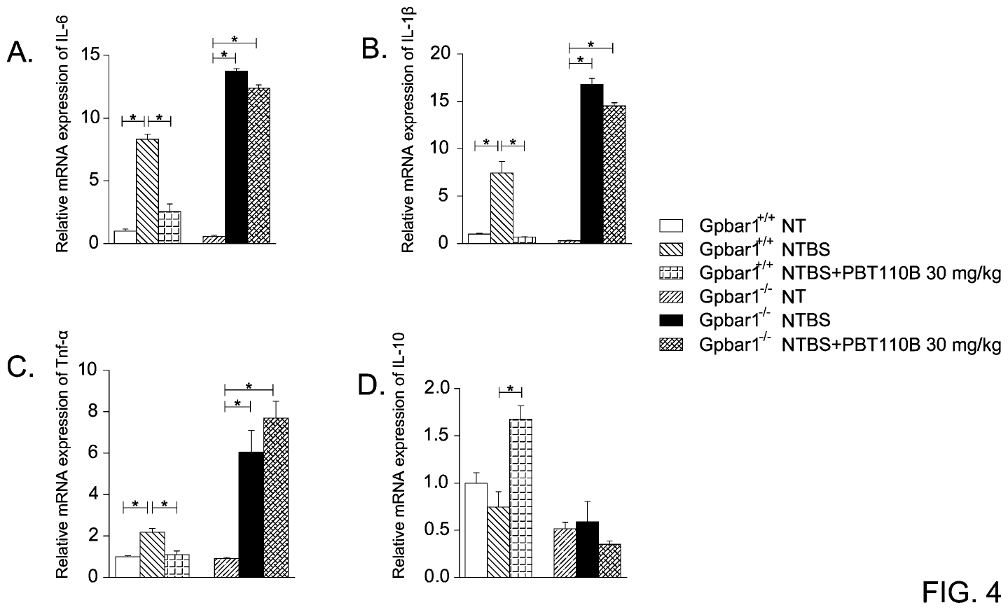

### BEST MODE FOR CARRYING OUT THE INVENTION

The following paragraphs provide definitions of the various chemical moieties of the compounds according to the invention and are intended to apply uniformly through-out the specification and claims unless an otherwise expressly set out definition provides a broader definition.

The term “alkyl”, as used herein, refers to saturated aliphatic hydrocarbon groups. Such term includes straight (unbranched) chains or branched chains.

Non-limiting examples of alkyl groups according to the invention are, for example, methyl, ethyl, propyl, isopropyl, n-butyl, iso-butyl, tert-butyl, n-pentyl, iso-pentyl, n-hexyl and the like.

The term “alkenyl”, as used herein, refers to unsaturated aliphatic hydrocarbon groups with at least one double bond. Such term includes straight (unbranched) chains or branched chains.

Non-limiting examples of alkenyl groups according to the invention are, for example, vinyl, 1-propenyl, 2-propenyl, 1- or 2-butenyl, and the like.

The term “alkynyl”, as used herein, refers to unsaturated aliphatic hydrocarbon groups with at least one triple bond. Such term includes straight (unbranched) chains or branched chains.

Non-limiting examples of alkynyl groups according to the invention are, for example, ethynyl, 1-propynyl, 2-propynyl, 1- or 2-butynyl, and the like.

The term “aryl”, as used herein, refers to a hydrocarbon consisting of an unsubstituted or substituted mono-, bi- or tricarbocyclic ring system, wherein the rings are fused together and at least one of the carbocyclic ring is aromatic. The term “aryl” means for example a cyclic aromatic such as a 6-membered hydrocarbon ring, a two six-membered fused hydrocarbon rings. Non-limiting examples of aryl groups are, for example, phenyl, alpha- or beta-naphthyl, 9,10-dihydroanthracenyl, indanyl, fluorenyl and the like. Aryl groups according to the present invention may be unsubstituted or substituted by one or more substituents as defined below.

Unless otherwise indicated, the term “substituted”, as used herein, means that one or more hydrogen atoms of the above-mentioned groups are replaced with another non-hydrogen atom or functional group, provided that normal valences are maintained and that the substitution results in a stable compound.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The compounds of Formula (I) may readily be isolated in association with solvent molecules by crystallization or evaporation of an appropriate solvent to give the corresponding solvates.

The compounds of Formula (I) may be in crystalline form. In certain embodiments, the crystalline forms of the compounds of Formula (I) are polymorphs.

The subject invention also includes isotopically-labelled compounds, which are identical to those recited in Formula (I), but differ on the fact that one or more atoms are replaced by an atom having an atomic mass or mass number different from the atomic mass or mass number usually found in nature. Examples of isotopes that can be incorporated into the compounds of the invention include isotopes of hydrogen, carbon, nitrogen, oxygen, such as ²H, ³H, ¹¹C, ¹³C, ¹⁴C, ¹⁵N, ¹⁷O, ¹⁸O.

Compounds of the present invention that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e. ³H, and carbon-14, i.e. ¹⁴C, isotopes are particularly preferred for their ease of preparation and detectability. ¹¹C isotope is particularly useful in PET (Positron Emission Tomography). Furthermore, substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. Isotopically-labelled compounds of Formula (I) of this invention can generally be prepared by carrying out the procedures disclosed in the Schemes and/or in the Examples below, by replacing a non-isotopically-labelled reagent with a readily available isotopically-labelled reagent.

Certain groups/substituents included in the present invention may be present as isomers. Accordingly, in certain embodiments, the compounds of Formula (I) may have axial asymmetries and, correspondingly, they may exist in the form of optical isomers such as an (R)-form, an (S)-form, and the like. The present invention includes within the scope all such isomers, including racemates, enantiomers and mixtures thereof.

In particular, within the scope of the present invention are included all stereoisomeric forms, including enantiomers, diastereoisomers, and mixtures thereof, including racemates, and the general reference to the compounds of Formula (I) includes all the stereoisomeric forms, unless otherwise indicated.

In general, the compounds of the invention should be interpreted as excluding those compounds (if any) which are so chemically unstable, either per se or in water, that they are clearly unsuitable for pharmaceutical use through all administration routes, whether oral, parenteral, or otherwise. Such compounds are known to the skilled chemist.

According to a first aspect of the invention, a compound of Formula (I):

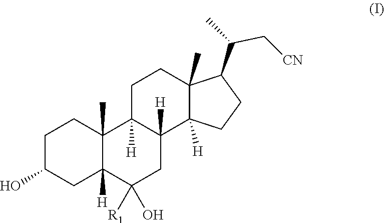

or pharmaceutically acceptable isomers or solvates thereof.

In the Compounds of Formula (I):

R₁ is selected from the group consisting of linear or branched C₁-C₆ alkyl, linear or branched C₂-C₆ alkenyl, linear or branched C₂-C₆ alkynyl, benzyl and aryl.

Surprisingly, the inventors have found that, although the hyodeoxycholic acid is a weak GPBAR1 agonist, slight modifications in the lateral chain and on ring B result in potent compounds able to transactivate GPBAR1.

According to an embodiment, R₁ is selected from the group consisting of methyl, ethyl, n-propyl, isopropyl, 2-propenyl, vinyl, phenyl, 4-pentenyl and benzyl.

According to a further embodiment, the compound of formula (I) can be selected from the group consisting of:

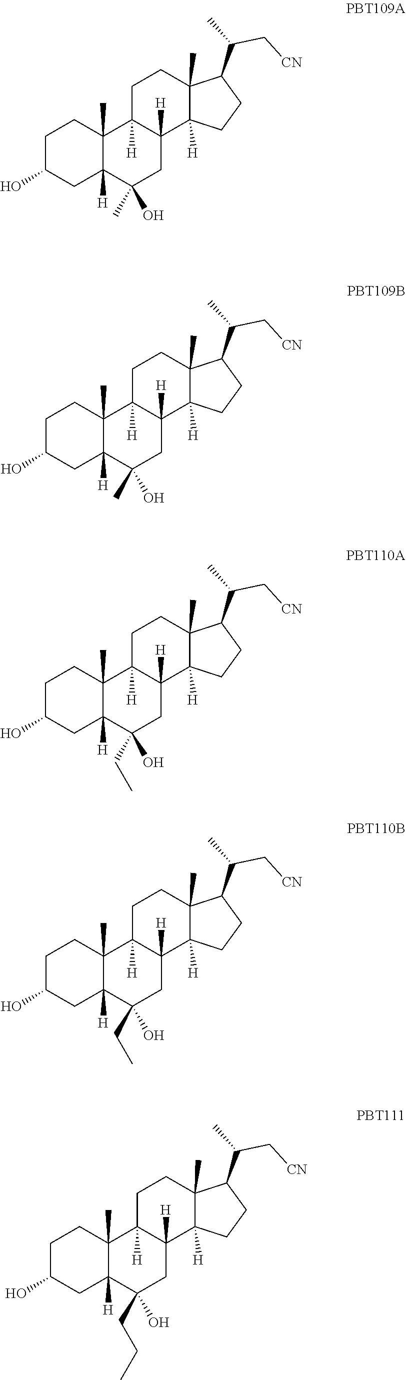

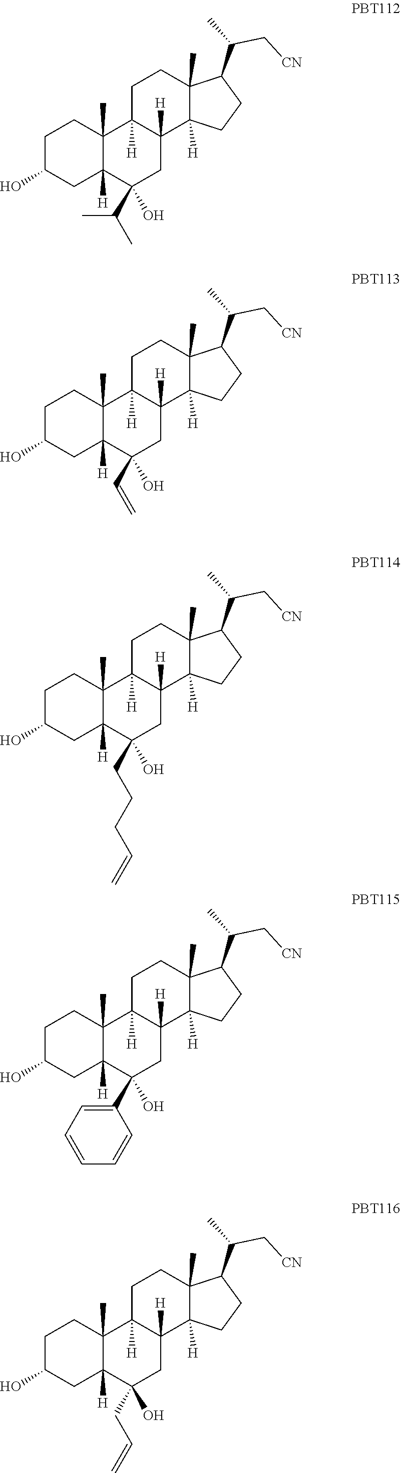

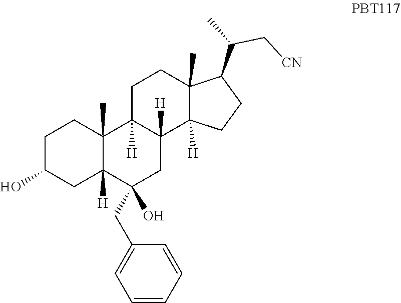

A second aspect of the present invention relates to a pharmaceutical composition comprising a compound of Formula (I) as disclosed above and at least one pharmaceutically acceptable excipient.

A person skilled in the art is aware of a whole variety of such excipient compounds suitable to formulate a pharmaceutical composition.

The compounds of the invention, together with a conventionally employed excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral administration (including subcutaneous and intravenous use).

Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Pharmaceutical compositions containing a compound of this invention can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention can be administered by a variety of routes including oral, rectal, subcutaneous, intravenous, intramuscular, intranasal and pulmonary routes. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampoules or syringes of the liquid compositions or pills, tablets, capsules or the like in the case of solid compositions.

Liquid forms suitable for oral administration may include a suitable aqueous or non-aqueous vehicle with buffers, suspending and dispensing agents, colorants, flavours and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel or corn starch; a lubricant such as magnesium stearate; a glidant such as colloidal silicon dioxide; a sweetening agent such as sucrose or saccharin; or a flavouring agent such as peppermint, methyl salicylate, or orange flavouring.

Injectable compositions are typically based upon injectable sterile or phosphate-buffered or other injectable carriers known in the art.

The pharmaceutical compositions may be in the form of tablets, pills, capsules, solutions, suspensions, emulsion, powders, suppository and as sustained release formulations.

If desired, tablets may be coated by standard aqueous or non-aqueous techniques. In certain embodiments, such compositions and preparations can contain at least 0.1 percent of active compound. The percentage of active compound in these compositions may, of course, be varied and may conveniently be between about 1 percent to about 60 percent of the weight of the unit. The amount of active compound in such therapeutically useful compositions is such that therapeutically active dosage will be obtained. The active compounds can also be administered intranasal as, for example, liquid drops or spray.

The tablets, pills, capsules, and the like may also contain a binder such as gum tragacanth, acacia, corn starch or gelatin; excipients such as calcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propyl parabens as preservatives, a dye and a flavoring agent such as cherry or orange flavor. To prevent breakdown during transit through the upper portion of the gastrointestinal tract, the composition be an enteric coated formulation.

Compositions for pulmonary administration include, but are not limited to, dry powder compositions consisting of the powder of a compound of Formula (I), and the powder of a suitable carrier and/or lubricant. The compositions for pulmonary administration can be inhaled from any suitable dry powder inhaler device known to a person skilled in the art.

Administration of the compositions is performed under a protocol and at a dosage sufficient to reduce the inflammation and pain in the subject. In some embodiments, in the pharmaceutical compositions of the present invention the active principle or active principles are generally formulated in dosage units. The dosage unit may contain from 0.1 to 1000 mg of a compound of Formula (I) per dosage unit for daily administration.

In some embodiments, the amounts effective for a specific formulation will depend on the severity of the disease, disorder or condition, previous therapy, the individual's health status and response to the drug. In some embodiments, the dose is in the range from 0.001% by weight to about 60% by weight of the formulation.

When used in combination with one or more other active ingredients, the compound of the present invention and the other active ingredient may be used in lower doses than when each is used singly.

Concerning formulations with respect to any variety of routes of administration, methods and formulations for the administration of drugs are disclosed in Remington's Pharmaceutical Sciences, 17th Edition, Gennaro et al. Eds., Mack Publishing Co., 1985, and Remington's Pharmaceutical Sciences, Gennaro A R ed. 20th Edition, 2000, Williams & Wilkins Pa., USA, and Remington: The Science and Practice of Pharmacy, 21st Edition, Lippincott Williams & Wilkins Eds., 2005; and in Loyd V. Allen and Howard C. Ansel, Ansel's Pharmaceutical Dosage Forms and Drug Delivery Systems, 10th Edition, Lippincott Williams & Wilkins Eds., 2014.

The above described components for orally administered or injectable compositions are merely representative.

The compounds of this invention can also be administered in sustained release forms or from sustained release drug delivery systems.

A third aspect of the present invention relates to a compound of Formula (I) as disclosed above, for the use as a medicament.

A compound of Formula (I) as disclosed above can be used in the prevention and/or treatment of a disorder selected from the group consisting of gastrointestinal disorders, liver diseases, cardiovascular diseases, atherosclerosis, metabolic diseases, metabolic disorders, infectious diseases, cancer, renal disorders, inflammatory disorders, and neurological disorders such as stroke.

In one embodiment, the liver disease is selected in the group consisting of chronic liver diseases including primary biliary cirrhosis (PBC), cerebrotendinous xanthomatosis (CTX), primary sclerosing cholangitis (PSC), drug induced cholestasis, intrahepatic cholestasis of pregnancy, parenteral nutrition associated cholestasis, bacterial overgrowth and sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant, liver regeneration, congenital hepatic fibrosis, granulomatous liver disease, intra- or extrahepatic malignancy, Wilson's disease, hemochromatosis, and alpha 1-antitrypsin deficiency.

In one embodiment, the gastrointestinal disease is selected in the group consisting of inflammatory bowel disease (IBD) (including Crohn's disease, ulcerative colitis and undetermined colitis), irritable bowel syndrome (IBS), bacterial overgrowth, acute and chronic pancreatitis, malabsorption, post-radiation colitis, and microscopic colitis.

In one embodiment, the renal disease is selected in the group consisting of diabetic nephropathy, hypertensive nephropathy, chronic glomerular disease, including chronic glomerulonephritis and chronic transplant glomerulopathy, chronic tubulointerstitial diseases and vascular disorders of the kidney.

In one embodiment, the cardiovascular disease is selected in the group consisting of atherosclerosis, dyslipidemia, hypercholesterolemia, hypertriglyceridemia, hypertension also known as arterial hypertension, inflammatory heart disease including myocarditis and endocarditis, ischemic heart disease stable angina, unstable angina, myocardial infarction, cerebrovascular disease including ischemic stroke, pulmonary heart disease including pulmonary hypertension, peripheral artery disease (PAD), also known as peripheral vascular disease (PVD) peripheral artery occlusive disease, and peripheral obliterative arteriopathy.

In one embodiment, the metabolic disease is selected in the group consisting of insulin resistance, metabolic syndrome, Type I and Type II diabetes, hypoglycemia, disorders of adrenal cortex including adrenal cortex insufficiency.

In one embodiment, metabolic disorder is selected in the group consisting of obesity and conditions associated to bariatric surgery.

In one embodiment, cancer is selected in the group of liver cancer, bile duct cancers, pancreatic cancer, gastric cancer, colon-rectal cancer, breast cancer, ovary cancer and condition associated with chemotherapy resistance.

In one embodiment, infectious disorder is selected in the group of human immunodeficiency associated disease (AIDS) and related disorders, virus B and Virus C infection.

In one embodiment, inflammatory disorder is selected in the group of rheumatoid arthritis, fibromyalgia, Syogren's syndrome, scleroderma, Behcet's syndrome, vasculitis and systemic lupus erythematosus.

Further characteristics of the present invention will result from the following description of some merely illustrative and non-limiting examples.

The following abbreviations are used in the attached examples.

Methanol (MeOH), sodium bicarbonate (NaHCO₃), ethyl acetate (EtOAc), dichloromethane (DCM), sodium sulphate (Na₂SO₄), sodium hydroxide (NaOH), water (H₂O), formic acid (HCOOH), perchloric acid (HClO₄), sodium nitrite (NaNO₂), deuterated chloroform (CDCl₃), deuterated methanol (CD₃OD), trifluoroacetic acid (TFA), dichloromethane (DCM), pyridinium dichromate (PDC), hour (h), room temperature (rt), retention time (t_{R}).

When not otherwise indicated, ¹H NMR was recorded on Varian Inova 400 and 500 MHz, using CD₃OD and CDCl₃ as solvents, and ¹³C NMR was recorded on Varian Inova 100 MHz, using CD₃OD and CDCl₃ as solvents.

### EXAMPLES

Preparation of Ketone 2.

Ketone 2 was prepared in a three-step procedure starting from HDCA, involving formylation at the hydroxyl groups in 3 and 6, the formation of C-23 nitrile on the side chain and subsequent oxidation at C-6, as previously described (Festa et al, J. Med. Chem. 2014, 57, 8477; Schteirgart, C. D. & Hofamann, A. E., J. Lipid Research, 1988, 29, 1387).

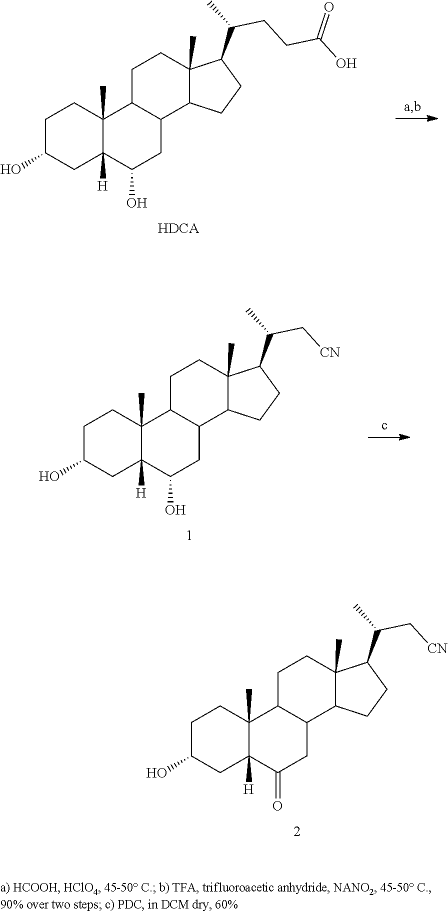 [SMILES: C[C@H](CCC(=O)O)[C@H]1CCC2C3C[C@H](O)[C@@H]4C[C@H](O)CC[C@]4(C)C3CC[C@@]21C ; C[C@H](CC#N)[C@H]1CCC2C3C[C@H](O)[C@@H]4C[C@H](O)CC[C@]4(C)C3CC[C@@]21C ; C[C@H](CC#N)[C@H]1CCC2C3CC(=O)[C@@H]4C[C@H](O)CC[C@]4(C)C3CC[C@@]21C]

### General Procedures

Step a-b). To a solution of hyodeoxycholic acid (HDCA) (2.0 g, 5.09 mmol) in 30 mL of 90% formic acid were added 300 μL of 70% perchloric acid at 0° C. The reaction was heated under stirring at 45-50° C. for 12 h. The temperature of the heating bath was lowered to 40° C., then 15 mL of acetic anhydride was added and the mixture was stirred for 15 min. The solution was cooled to room temperature, poured into 50 mL of water and extracted with diethyl ether (3×50 mL). The organic layers were washed with saturated NaHCO₃ solution (50 mL) and water to neutrality, dried over Na₂SO₄, and evaporated to give 2.04 g of a crude residue, that was subjected to the next step reaction without any purification. Formyl HDCA (2.04 g, 4.55 mmol), 29 mL of cold trifluoroacetic acid (TFA), and 3.8 mL (27.3 mmol) of trifluoroacetic anhydride were stirred at 0° C. until dissolution. Sodium nitrite (942 mg, 13.6 mmol) was added at the solution. The reaction mixture was stirred first at 0-5° C. for 1 h, then at 45-50° C. for 3 h. When the reaction was completed, it was neutralized with NaOH 6 N, in order to remove both the acid reagent used, that the protecting group (formyl group) at C3 and C6. Then the deprotected product was extracted with 50 mL of diethyl ether (3×50 mL), followed by washing with brine and dried over anhydrous Na₂SO₄. The ether was removed under reduced pressure to afford 5.4 g of a crude product. Purification by silica gel eluting with Hexane-EtOAc (40:60) gave the nornitrile 1 as a white solid (1.65 g, 4.6 mmol, 90% over two steps).

3α, 6α-dihydroxy-24-nor-5ββ-cholane-23-nitrile (compound 1): C₂₃H₃₇NO₂

¹H NMR (400 MHz, CDCl₃): δ 4.07 (1H, dt, J=4.4, 11.0 Hz, H-6β) 3.63 (1H, m, H-3β2.37 (1H, dd, J=3.5, 16.6 Hz, H-22), 2.22 (1H, dd, J=7.4, 16.6 Hz, H-22), 1.16 (3H, d, J=6.6 Hz, Me-21), 0.92 (3H, s, Me-19), 0.67 (3H, s, Me-18)

¹³C NMR (100 MHz, CDCl₃): δ119.0, 71.5, 68.0, 55.9, 54.9, 48.4, 42.9, 39.7, 39.6, 35.9, 35.5, 34.9, 34.8, 33.6, 30.3, 29.2, 28.1, 24.7, 24.1, 23.4, 20.7, 19.3, 12.1

Step c). The nornitrile 1 (1.65 g, 4.6 mmol) was subjected to oxidation at C6. After solubilisation in dichloromethane dry (20 mL), pyridinium dichromate (2 g, 9.2 mmol) was added, and the reaction mixture was stirred at room temperature for 5 hours. When the substrate was consumed, the solvent was dried under reduced pressure; the product was dissolved in EtOAc, filtered through celite under vacuum, and then evaporated to furnish 1.5 g of a crude residue that was purified by flash chromatography using 65:35 Hexane-EtOAc as mobile phase giving compound 2 (985 mg, 6β% yield).

3α-hydroxy-6-keto-24-nor-5β-cholane-23-nitrile (compound 2): C₂₃H₃₅NO₂

¹H NMR (500 MHz, CD₃OD): δ3.54 (1H, m, H-3), 2.47 (dd, J=3.2, 16.9 Hz, H-22), 2.36 (dd, J=7.0, 16.9 Hz, H-22), 1.17 (3H, d, J=6.3 Hz, Me-21), 0.84 (3H, s, Me-19), 0.74 (3H, s, Me-18);

¹³C NMR (100 MHz, CDCl₃): δ 213.5, 118.8, 70.0, 59.4, 56.5, 54.7, 43.1, 42.7, 39.9, 39.2, 37.9, 36.9, 34.8, 34.3, 33.4, 29.8, 27.9, 24.7, 23.9, 23.1, 20.7, 19.2, 12.0.

### Example 1. Preparation of 6-Substituted Derivatives: PBT109A-B, PBT110A-B, PBT111-117

Starting from intermediate 2, the same synthetic procedure was performed to obtain the different C6-substituted products. Ketone 2 was treated with nine different magnesium halides via Grignard reaction to afford compounds PBT109A-B, PBT110A-B, PBT111-117.

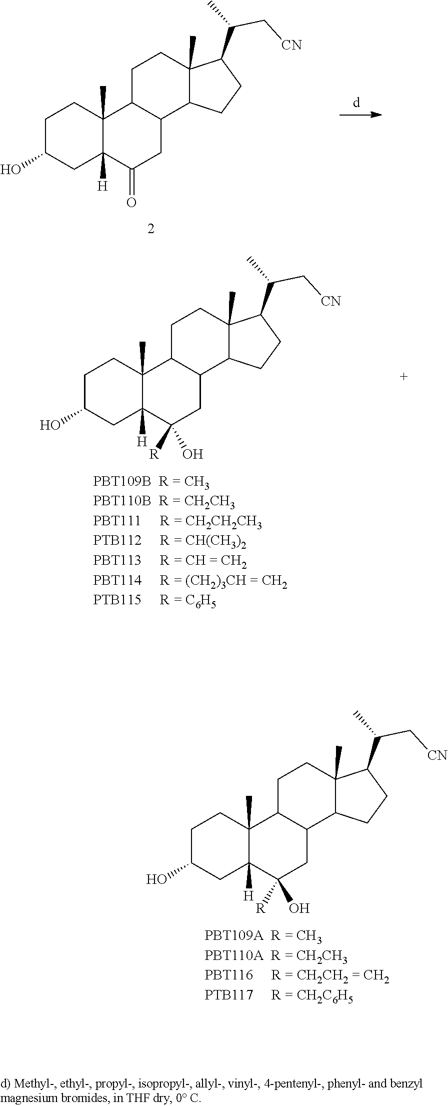

The ketone 2 was dried with toluene and argon under reduced pressure, in order to remove any trace of water. Then it was dissolved in THF dry at 0° C., the Grignard reagents (10 mol eq) were added and the reaction were stirred under argon for 4 h. When the reactions were completed, 1 mL of NH₄Cl solution (1M) was added, the mixture was stirred for ten minutes and then extracted with H₂O/EtOAc (20 mL) for three times. The organic layers were dried over Na₂SO₄, filtered and concentrated under reduced pressure, to give derivatives PBT109A-B, PBT110A-B, PBT111-117.

### Example 1A. Preparation of 3α, 6β-dihydroxy-6α-methyl-24-nor-5β-cholan-23-nitrile (PBT109A) and 3α,6α-dihydroxy-6β-methyl-24-nor-5β-cholan-23-nitrile (PBT109B)

The treatment of ketone 2 (50 mg, 0.14 mmol) with methyl magnesium bromide furnished a mixture of PBT109A and PBT109B that was efficiently separated by HPLC on a Luna Omega Polar C18 (5 μm; 4.6 mm i.d.×250 mm) with MeOH/H₂O (75:25) as eluent (flow rate 1 mL/min) giving PBT109A (13 mg, t_{R}=10.5 min) and PBT109B (22 mg, t_{R}=18.2 min).

3α,6β-dihydroxy-6α-methyl-24-nor-5β-cholan-23-nitrile (PBT109A): C₂₄H₃₉NO₂

¹H NMR (500 MHz, CD₃OD): δ 3.47 (1H, m, H-3δ), 2.46 (1H, dd, J=3.3, 17.0 Hz, H-22), 2.34 (1H, dd, J=7.0, 17.0 Hz, H-22), 1.15 (3H, d, J=6.7 Hz, Me-21), 1.14 (3H, s, Me-19), 1.12 (3H, s, Me-24), 0.74 (3H, s, Me-18);

¹³C NMR (100 MHz, CD₃OD): δ 120.3, 74.3, 72.3, 57.4, 56.5, 53.5, 44.0, 41.8, 41.3, 41.1, 37.5, 36.7, 35.8, 34.7, 33.3, 30.8, 29.0, 27.2, 25.2, 25.0, 21.6, 19.6, 12.5

3α,6α-dihydroxy-6β-methyl-24-nor-5β-cholan-23-nitrile (PBT109B): C₂₄H₃₉NO₂

¹H NMR (500 MHz, CD₃OD): δ 3.47 (1H, m, H-3(3), 2.45 (1H, dd, J=3.4, 16.9 Hz, H-22), 2.33 (1H, dd, J=7.0, 16.9 Hz, H-22), 1.40 (3H, s, Me-24), 1.15 (3H, d, J=6.7 Hz, Me-21), 0.99 (3H, s, Me-19), 0.74 (3H, s, Me-18)

¹³C NMR (100 MHz, CD₃OD): δ 120.3, 72.9, 72.4, 57.5, 56.5, 54.9, 44.0, 41.9, 41.5, 41.0, 37.0, 35.3, 34.7, 33.9, 32.0, 31.4, 28.9, 27.5, 25.3, 25.0, 21.7, 19.6, 12.5

### Example 1B. Preparation of 3α,6β-dihydroxy-6α-ethyl-24-nor-5β-cholan-23-nitrile (PBT110A) and 3α,6α-dihydroxy-6β-ethyl-24-nor-5β-cholan-23-nitrile (PBT10B)

The treatment of ketone 2 (300 mg, 0.84 mmol) with ethyl magnesium bromide furnished a mixture of PBT110A and PBT110B that was efficiently separated by HPLC on a Luna C18 (10 μm; 4.6 mm i.d.×250 mm) with MeOH/H₂O (80:20) as eluent (flow rate 3 mL/min) giving PBT110A (35 mg, t_{R}=11.3 min) and PBT110B (150 mg, t_{R}=22.1 min).

3α6β-dihydroxy-6α-ethyl-24-nor-5β-cholan-23-nitrile (PBT110A): C₂₅H₄₁NO₂

¹H NMR (500 MHz, CD₃OD): δ 3.47 (1H, m, H-3β), 2.46 (1H, dd, J=3.5, 16.7 Hz, H-22), 2.34 (1H, dd, J=6.9, 16.7 Hz, H-22), 1.16 (3H, d, J=6.6 Hz, Me-21), 1.15 (3H, s, Me-19), 0.88 (3H, t, J=7.3 Hz, Me-25), 0.74 (3H, s, Me-18)

¹³C NMR (100 MHz, CD₃OD): δ 120.4, 75.8, 72.2, 57.4, 56.4, 50.3, 43.9, 42.3, 41.0, 40.4, 37.5, 36.8, 35.4, 35.2, 34.6, 33.2, 30.8, 29.0, 27.2, 25.1, 24.8, 21.6, 19.5, 12.2, 6.4

3α-dihydroxy-6β-ethyl-24-nor-5β-cholan-23-nitrile (PBT110B): C₂₅H₄₁NO₂

¹H NMR (500 MHz, CD₃OD): δ 3.48 (1H, m, H-3β), 2.46 (1H, dd, J=3.7, 16.9 Hz, H-22), 2.33 (1H, dd, J=7.0, 16.9 Hz, H-22), 1.16 (3H, d, J=6.7 Hz, Me-21), 1.01 (3H, s, Me-19), 0.92 (3H, t, J=7.4 Hz, Me-25), 0.73 (3H, s, Me-18)

¹³C NMR (100 MHz, CD₃OD): δ 120.3, 74.6, 72.3, 57.6, 56.5, 51.3, 44.1, 42.1, 41.0, 39.9, 38.1, 37.1, 35.5, 34.9, 34.7, 33.9, 31.3, 28.9, 26.9, 25.0 (2C), 21.7, 19.6, 12.4, 8.1

### Example 1C. Preparation of 3α,6α-dihydroxy-6β-propyl-24-nor-5β-cholan-23-nitrile (PBT111)

An analytic sample of crude reaction with propyl magnesium bromide was purified by HPLC on a Luna Omega Polar C18 (5 μm; 4.6 mm i.d.×250 mm) with MeOH/H₂O (78:22) as eluent (flow rate 1 mL/min) giving PBT111 (t_{R}=27.0 min): C₂₆H₄₃NO₂

¹H NMR (500 MHz, CD₃OD): δ 3.48 (1H, m, H-3β), 2.46 (1H, dd, J=3.7, 16.9 Hz, H-22), 2.33 (1H, dd, J=7.2, 16.9 Hz, H-22), 1.15 (3H, d, J=6.8 Hz, Me-21), 1.00 (3H, s, Me-19), 0.92 (3H, t, J=7.2 Hz, Me-26), 0.74 (3H, s, Me-18).

¹³C NMR (100 MHz, CD₃OD): δ 120.3, 74.5, 72.4, 57.5, 56.5, 51.4, 45.9, 43.9, 42.3, 40.9, 40.5, 38.1, 37.0, 35.1, 34.6, 33.9, 31.4, 28.9, 26.8, 25.0, 25.3, 21.6, 19.6, 17.7, 15.0, 12.5

### Example 1D. Preparation of 3α, 6α-dihydroxy-6β-isopropyl-24-nor-5β-cholane-23-nitrile (PBT112)

An analytic sample of crude reaction with isopropyl magnesium bromide was purified by HPLC on a Luna Omega Polar C18 (5 μm; 4.6 mm i.d.×250 mm) with MeOH/H₂O (78:22) as eluent (flow rate 1 mL/min) giving PBT112 (t_{R}=40.7 min): C₂₆H₄₃NO₂

¹H NMR (400 MHz, CD₃OD): δ 3.50 (1H, m, H-3β), 2.46 (1H, dd, J=3.7, 16.8 Hz, H-22), 2.35 (1H, dd, J=7.1, 16.8 Hz, H-22), 1.15 (3H, d, J=6.6 Hz, Me-21), 1.03 (3H, s, Me-19), 0.95 (3H, d, J=6.7 Hz, Me-25), 0.89 (3H, d, J=6.8 Hz, Me-26), 0.74 (3H, s, Me-18).

### Example 1E. Preparation of 3α, 6α-dihydroxy-6β-vinyl-24-nor-5β-cholane-23-nitrile (PBT113)

An analytic sample of crude reaction with vinyl magnesium bromide was purified by HPLC on a Synergi Fusion C18 (4 μm; 4.6 mm i.d.×250 mm) with MeOH/H₂O (75:25) as eluent (flow rate 1 mL/min) giving PBT113 (t_{R}=20.0 min): C₂₅H₃₉NO₂

Selected ¹H NMR (400 MHz, CD₃OD): 6.09 (1H, dd, J=17.7, 10.8 Hz, H-24), 5.22 (1H, dd, J=17.7, 1.0 Hz, H-25), 5.06 (1H, d, J=10.8, 1.0 Hz, H-25), 3.51 (1H, m, H-3(3), 2.45 (1H, dd, J=3.7, 16.9 Hz, H-22), 2.34 (1H, dd, J=7.1, 16.9 Hz, H-22), 1.15 (3H, d, J=6.6 Hz, Me-21), 0.90 (3H, s, Me-19), 0.71 (3H, s, Me-18)

¹³C NMR (100 MHz, CD₃OD): δ 148.7, 120.2, 112.3, 73.2, 72.4, 57.5, 56.4, 54.1, 43.8, 41.7, 40.9, 37.6, 37.5, 37.3, 35.2, 34.8, 33.3, 31.3, 28.9, 26.7, 25.2, 25.0, 21.2, 19.6, 12.5

### Example 1F. Preparation of 3α, 6α-dihydroxy-6β-(4-Pentenyl)-24-Nor-5β-Cholane-23-Nitrile (PBT114)

An analytic sample of crude reaction with pent-4-en-1-yl-magnesium bromide was purified by HPLC on a Synergi Fusion C18 (4 μm; 4.6 mm i.d.×250 mm) with MeOH/H₂O (75:25) as eluent (flow rate 1 mL/min) giving PBT114 (t_{R}=20.14 min): C₂₈H₄₅NO₂

¹H NMR (500 MHz, CD₃OD): δ 5.84 (1H, m, H-27), 5.02 (1H, dd, J=17.1, 1.9 Hz, H-28), 4.94 (1H, dd, J=10.2, 1.9, H-28), 3.49 (1H, m, H-3(3), 2.45 (1H, dd, J=3.7, 16.8 Hz, H-22), 2.33 (1H, dd, J=7.2, 16.8 Hz, H-22), 1.14 (3H, d, J=6.6 Hz, Me-21), 0.99 (3H, s, Me-19), 0.73 (3H, s, Me-18)

¹³C NMR (100 MHz, CD₃OD): δ140.2, 120.4, 114.9, 74.6, 72.4, 57.3, 56.4, 51.6, 43.8, 42.7, 42.3, 40.9, 40.4, 37.8, 36.8, 35.3, 34.9, 34.7, 33.8, 31.4, 30.6, 28.9, 26.9, 25.2, 24.9, 21.5, 19.6, 12.4

### Example 1G. Preparation of 3α, 6α-dihydroxy-6β-phenyl-24-nor-5β-cholane-23-nitrile (PBT115)

An analytic sample of crude reaction with phenyl magnesium bromide was purified by HPLC on a Luna Omega Polar C18 (5 μm; 4.6 mm i.d.×250 mm) with MeOH/H₂O (78:22) as eluent (flow rate 1 mL/min) giving PBT115 (t_{R}=21.7 min) C₂₉H₄₁NO₂

¹H NMR (400 MHz, CD₃OD): δ 7.51 (1H, m, H-4′), 7.34 (2H, br t, J=7.5 Hz, H-3′), 7.23 (2H, t, J=7.2 Hz, H-2′), 3.60 (1H, m, H-3β), 2.45 (1H, dd, J=3.6, 16.8 Hz, H-22), 2.36 (1H, dd, J=7.1, 16.8 Hz, H-22), 1.14 (3H, d, J=6.7 Hz, Me-21), 0.70 (3H, s, Me-18), 0.20 (3H, s, Me-19)

¹³C NMR (100 MHz, CD₃OD): δ 149.8, 129.9, 129.5 (2C), 128.2 (2C), 120.0, 72.5, 74.5, 57.9, 56.7, 52.5, 44.2, 42.2, 41.0, 38.0, 37.6, 36.0, 35.6, 34.9, 33.7, 31.2, 29.2, 25.9, 25.4, 25.2, 21.7, 19.8, 12.7

### Example 1H. Preparation of 3α,6β-dihydroxy-6α-(2-propenyl)-24-nor-5β-cholan-23-nitrile (PBT116)

An analytic sample of crude reaction with allyl magnesium bromide was purified by HPLC on a Luna Omega Polar C18 (5 μm; 4.6 mm i.d.×250 mm) with MeOH/H₂O (68:32) as eluent (flow rate 1 mL/min) giving PBT116 (t_{R}=33.0 min): C₂₆H₄₁NO₂

¹H NMR (500 MHz, CD₃OD): δ5.95 (1H, m, H-25), 5.10 (1H, dd, J=2.0, 9.8, H-26), 5.08 (1H, dd, J=2.0, 17.0, H-26), 3.44 (1H, m, H-3β2.46 (1H, dd, J=3.7, 16.9 Hz, H-22), 2.34 (1H, dd, J=7.1, 16.9 Hz, H-22), 1.16 (3H, d, J=6.6 Hz, Me-21), 1.14 (3H, s, Me-19), 0.74 (3H, s, Me-18)

¹³C NMR (100 MHz, CD₃OD): δ 135.0, 120.3, 118.2, 75.7, 72.1, 57.5, 56.5, 51.2, 47.9, 44.0, 42.3, 41.1, 40.7, 37.6, 36.7, 34.9, 34.7, 30.8, 33.2, 29.0, 27.2, 25.2, 25.0, 21.6, 19.6, 12.5

### Example 1G. Preparation of 3α,6β-dihydroxy-6α-benzyl-24-nor-5β-cholane-23-nitrile (PBT117)

An analytic sample of crude reaction with benzyl magnesium bromide was purified by HPLC on a Luna Omega Polar C18 (5 μm; 4.6 mm i.d.×250 mm) with MeOH/H₂O (78:22) as eluent (flow rate 1 mL/min) giving PBT117 (t_{R}=32.7 min) C₃₀H₄₃NO₂

¹H NMR (400 MHz, CD₃OD): δ 7.22 (4H, ovl, H-2′, H-3′, H-5′ and H-6′), 7.14 (1H, m, H-4′), 3.48 (1H, m, H-3β3.37 (1H, d, J=13.8 Hz, H-24), 2.65 (1H, d, J=13.8 Hz, H-24), 2.46 (1H, dd, J=3.7, 16.9 Hz, H-22), 2.32 (1H, dd, J=7.1, 16.9 Hz, H-22), 1.15 (3H, ovl, Me-21), 1.15 (3H, s, Me-19), 0.80 (3H, s, Me-18)

¹³C NMR (100 MHz, CD₃OD): δ 139.7, 132.2 (2C), 128.6 (2C), 127.0, 120.3, 74.6, 72.4, 57.4, 56.4, 54.5, 48.2, 43.9, 42.2, 40.9, 38.2, 37.4 (2C), 34.8, 34.7, 34.1, 31.4, 28.9, 28.4, 25.2, 25.0, 21.7, 19.6, 12.5.

### Example 2. Biological Activities

Selected compounds were tested in vitro on FXR and TGR5/GPBAR1 receptors (Table 1) using a whole cell model transfected with a reporter genes. The activity was evaluated in comparison with chenodeoxycholic acid (CDCA) and TLCA.

CDCA is a primary bile acid that functions as an endogenous ligand for FXR, while TLCA is a physiological ligand for TGR5/GPBAR1. In this assay, HepG2 cells (a liver-derived cell line) were cultured at 37° C. in minimum essential medium with Earl's salts containing 10% fetal bovine serum (FBS), 1% L-glutamine, and 1% penicillin/streptomycin. HEK-293T cells were cultured at 37° C. in D-MEM containing 10% fetal bovine serum (FBS), 1% L-glutamine, and 1% penicillin/streptomycin. The transfection experiments were performed using Fugene HD according to manufactured specifications. Cells were plated in a 24-well plate at 5×10⁴ cells/well. For FXR mediated transactivation, HepG2 cells were transfected with 100 ng of pSG5-FXR, 100 ng of pSG5-RXR, 100 ng of pGL4.70 a vector encoding the human Renilla gene and 250 ng of the reporter vector p(hsp27)-TK-LUC containing the FXR response element IR1 cloned from the promoter of heat shock protein 27 (hsp27).

For GPBAR1 mediated transactivation, HEK-293T cells were transfected with 200 ng of pGL4.29, a reporter vector containing a cAMP response element (CRE) that drives the transcription of the luciferase reporter gene luc2P, with 100 ng of pCMVSPORT6-human GPBAR1, and with 100 ng of pGL4.70 a vector encoding the human Renilla gene. In control experiments HEK-293T cells were transfected only with vectors pGL4.29 and pGL4.70 to exclude any possibility that compounds could activate the CRE in a GPBAR1 independent manner. At 24 h post-transfection, cells were stimulated for 18 h with 10 μM TLCA as a control agent or putative GPBAR1 agonists as the same concentration. After treatments, cells were lysed in 100 μL of lysis buffer (25 mM Tris-phosphate, pH 7.8; 2 mM DTT; 10% glycerol; 1% Triton X-100), and 20 μL of cellular lysate was assayed for luciferase activity using the luciferase assay system. Luminescence was measured using Glomax 20/20 luminometer. Luciferase activities were normalized against Renilla activities. Antagonism against FXR of GPBAR1/TGR5 was measured as percent of activity in transactivation assay suing activity of TLCA as example of agonism.

THP-1, a human monocytic cell line derived from an acute monocytic leukemia patient, were cultured and maintained at 37° C. and 5% CO₂ in RPMI with 10% FBS, 1% glutamine and 1% penicillin/streptomycin. THP1 cells (1.5×10⁶) were serum starved for 6 h and then classically activated with LPS (1 ng/ml, L2880; Sigma-Aldrich), and exposed or not to GPBAR1 agonists previously tested in transactivation assay.

The data on the activity of certain compounds of the invention on TGR5/GPBAR1 are described in the following table 1. In this table, activities for compounds of the invention on GPBAR1 was compared to those of the reference compound TLCA. For % of activity, each compound was tested at the concentration of 10 microM and transactivation activity of TLCA on CRE (i.e. TGR5/GPBAR1) was considered equal to 100%. For selectivity toward FXR, activity was assessed in HepG2 cells transfected with an FXR responsive element (IR1) cloned upstream to the luciferase gene. For calculation of efficacy data, maximal transactivation of IR1 caused by each compound (10 μM) was compared to maximal transactivation caused by CDCA (10 μM). GPBAR1/FXR selectivity calculated as ratio between % of activity toward GPBAR1 and % of activity toward FXR. Each experiment was conducted in triplicate.

TABLE 1

| | GPBAR1 | FXR | | |
|---|---|---|---|---|
| | (% of | (% of | | |
| Compounds | activity in | activity in | | |
| of | comparison | comparison | | |
| formula | to 10 μM | to 10 μM | GPBAR1 | GPBAR1/FXR |
| (I) | TLCA) | CDCA) | EC50 μM | selectivity |
| PBT109A | 83.4 ± 2.4 | 5.5 ± 0.7 | 0.57 | 15.1 |
| PBT109B | 95.4 ± 0.24 | 6.4 ± 0.23 | 6.9 | 14.9 |
| PBT110A | 65.9 ± 1.69 | 6.6 ± 0.065 | 0.66 | 9.9 |
| PBT110B | 101.2 ± 4.1 | 12.3 ± 0.47 | 0.3 | 8.2 |
| PBT111 | 67.1 ± 2.1 | 3.1 ± 0.29 | 1.2 | 21.6 |
| PBT112 | 22.2 ± 0.13 | 4.3 ± 0.31 | 2.78 | 5.16 |
| PBT113 | 62.5 ± 2.18 | 2.2 ± 0.5 | 2.26 | 28.4 |
| PBT114 | 90.1 ± 1.35 | 1.6 ± 0.05 | 0.78 | 56.31 |
| PBT115 | 38.8 ± 1.79 | 18.2 ± 1.08 | 4.58 | 2.13 |
| PBT117 | 8.3 ± 0.015 | 4.4 ± 0.49 | 9.33 | 1.88 |

As shown in Table 1, selected compounds belonging to Formula I were effective in transactivating GPBAR1, exerting a minimal effect on FXR receptor. A selected example of potent and selective GPBAR1 agonist is PBT110B with an efficacy of 101.2% with respect to TLCA and an EC₅₀ value of 0.3 μM.

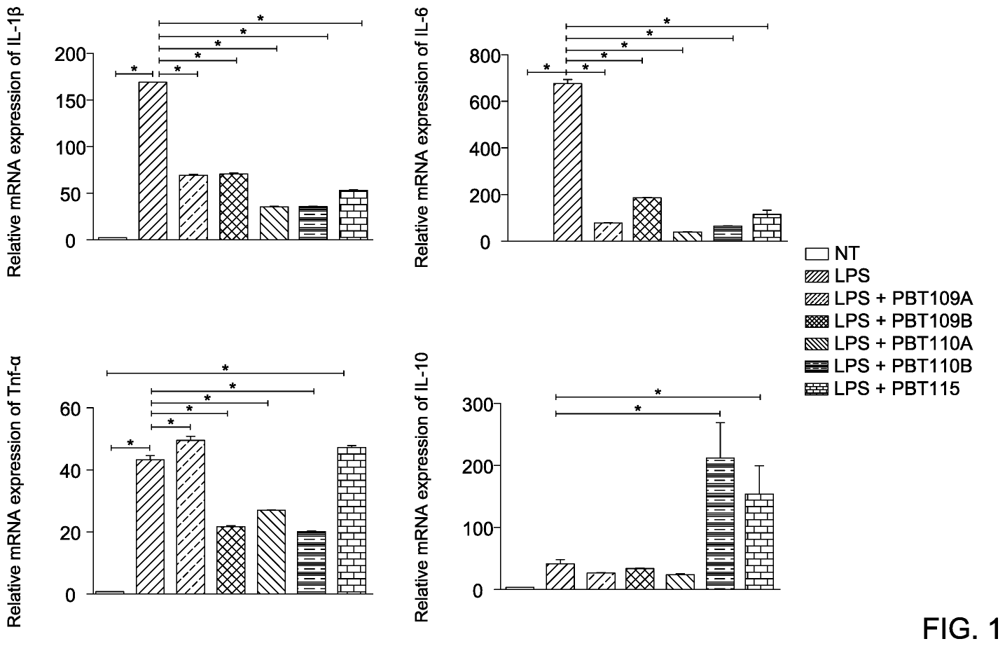

Furthermore, selected compounds exert in vitro activity on GPBAR1-target genes reducing the production of pro-inflammatory cytokines (IL-6, TNF-α, IL-1β, IL-10) in THP1 monocytic/macrophage cells primed with LPS and co-incubated with or without the selected compounds at 10 μM (FIG. 1).

FIG. 1 shows the results of relative mRNA expression of pro- and anti-inflammatory genes, IL-1β, IL-6, TNF-α, IL-10, assessed by Real-Time PCR in THP1 cells. Values are normalized relative to GAPDH mRNA and are expressed as mean±SEM (n=3); #p<0.05.

As shown in FIG. 1, PBT110B significantly reduced the production of pro-inflammatory cytokines (IL-6, TNF-α, IL1-β) in THP1 cells. Additionally, exposure to PBT110B increased the expression of anti-inflammatory gene IL-10, that is a specific target of GPBAR1.

Because macrophages are one of the most abundant leucocytes in the intestinal mucosa and are implicated in the pathogenesis of IBDs (Inflammatory bowel diseases), the effect of PBT110B was investigated in a TNBS-induced colitis (2,4,6-trinitrobenzene sulfonic acid-induced) in Gpbar1+/+ and Gpbar1−/− mice. Gpbar1+/+ and Gpbar1−/− mice were treated with TNBS alone or in combination PBT110B (30 mg/kg).

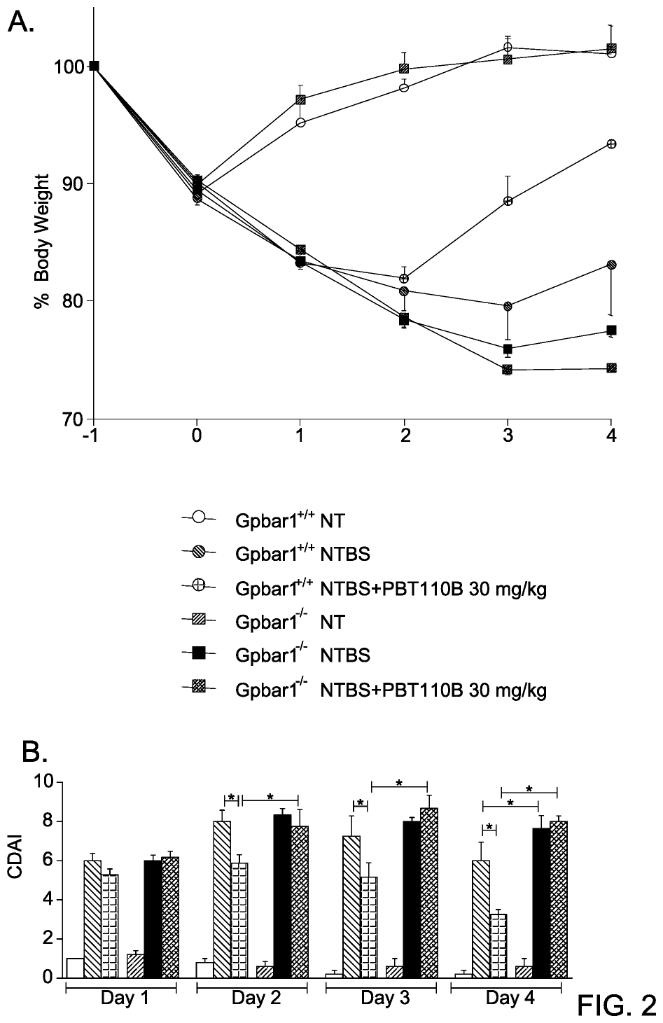

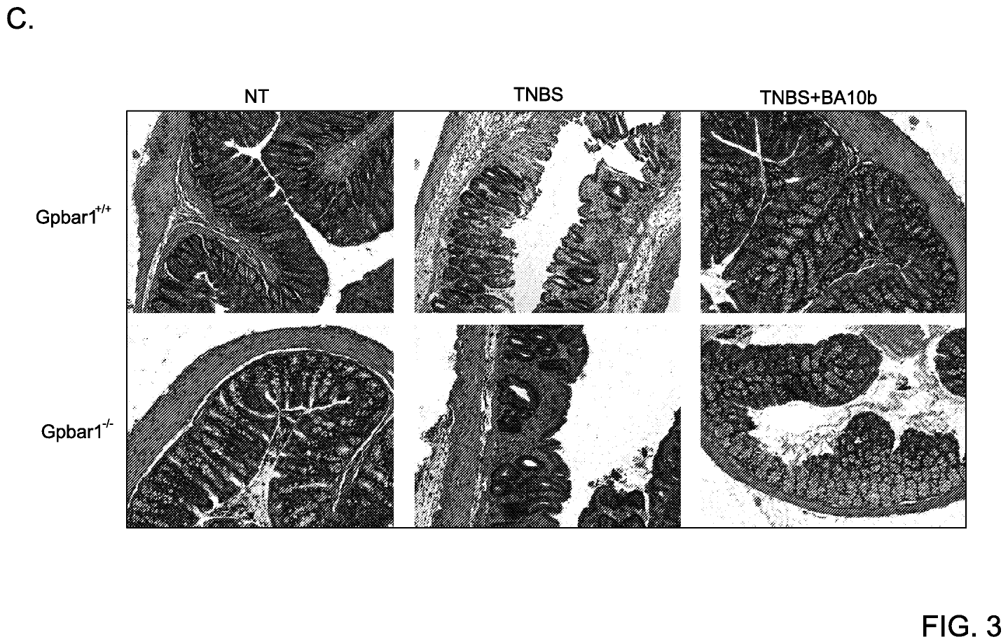

The development and the severity of colitis assessed by body weight lost, CDAI and histological sections (FIG. 2A-C), was attenuated by treatment with PBT110B in Gpbar1+/+ mice, that failed to do the same in the Gpbar1−/− mice. In addition, the expression of pro- and anti-inflammatory cytokines in the colon of these animals (FIG. 3) was analyzed. Gpbar1^{+/+} and Gpbar1^{-/-} mice were treated with TNBS alone or in combination PBT110B (30 mg/kg). Values are normalized relative to Gapdh mRNA and are expressed as mean±SEM (n=5); ^{#}p<0.05.

As expected, TNBS induced an increase in the expression of pro-inflammatory cytokines Il-6, Il-1β, TNF-α both in wild-type mice and in Gpbar1−/− mice (FIG. 3). On the contrary, PBT110B was able to revert this inflammatory pattern by reducing the expression of pro-inflammatory cytokines and also increasing the expression of the anti-inflammatory cytokine IL-10. These beneficial effects were lost in mice lacking the receptor.

Animals and Colitis Protocols

GPBAR1 null mice on C57BL/6NCrl background, and C57BL/6NCrl congenic littermates were originally donated by Dr. Galya Vassileva (Schering-Plough Research Institute, Kenilworth). The colonies were maintained in the animal facility of University of Perugia. Mice were housed under controlled temperatures (22° C.) and photoperiods (12:12-hour light/dark cycle), allowed unrestricted access to standard mouse chow and tap water and allowed to acclimate to these conditions for at least 5 days before inclusion in an experiment. The study was conducted in agreement with the Italian law and the protocol was approved by ethical committee of University of Perugia and by a National Committee of Italian Ministry of Health permit n° 1126/2016-PR. The health and body conditions of the animals were monitored daily by the veterinarian in the animal facility. The study protocol caused minor suffering, however, animals that lost more than 25% of the initial body weight were euthanized. Colitis was induced in mice by TNBS. Mice were fasted for 12 h (day −1) and the day after (day 0) mice were anesthetized, and a 3.5 F catheter inserted into the colon such that the tip was 3 cm proximal to the anus. To induce colitis, 1 mg of 2,4,6-trinitrobenzenesulfonic acid TNBS (Sigma Chemical Co, St Louis, Mo.) in 50% ethanol was administered via catheter into the lumen using a 1 ml syringe (injection volume of 200 μL); control mice received 50% ethanol alone. Animals were monitored daily. At the end of the experiments, the surviving mice were sacrificed and the colon was excised, weighed, and evaluated for macroscopic damage. In some groups of mice, PBT110B dissolved in nitrocellulose 1% was administered daily also by o.s. at the concentration of 30 mg/kg of body weight

The severity of colitis was measured each day for each mouse by assessing the body weight, the fecal blood and stool consistency. Each parameter was scored from 0 to 4 and the sum represents the Colitis Disease Activity Index (CDAI). The scoring system was as following: percent of body weight loss: none=0; 1-5%=1; 5-10%=2; 10-20%=3; >20%=4. Stool consistency: normal=0; soft but still formed=1; very soft=2; diarrhea=3; liquid stools that stick to the anus or anal occlusion=4. Fecal blood: none=0; visible in the stool=2; severe bleeding with fresh blood around the anus and very present in the stool=4.

Histology

Samples of distal colon (2-3 cm from the anus) were fixed in buffered formalin, cut into 5-μm-thick sections (150 μm between each section, four to eight per fragment per colon), and stained with H&E.

Reverse Transcription of mRNA and Real-Time PCR

Colon samples were immediately frozen in liquid nitrogen and stored at −80° C. until used, mechanically homogenated with the aid of a pestle, and the obtained materials re-suspended in 1 ml of Trizol (Thermo Scientific™). THP1 cells were spin-dried at 1300 rpm and then the pellets were re-suspended in 1 ml of Trizol (Thermo Scientific™). The RNA was extracted according to the manufacturer's protocol. After purification from genomic DNA by DNase-I treatment (Thermo Scientific™), 1 μg of RNA from each sample was reverse-transcribed using random hexamer primers with Superscript-II (Thermo Scientific™) in a 20 μL reaction volume; 10 ng cDNA were amplified in a 20 μL solution containing 200 nM of each primer and 10 μL of SYBR Select Master Mix (Thermo Scientific™). All reactions were performed in triplicate, and the thermal cycling conditions were as follows: 3 min at 95° C., followed by 40 cycles of 95° C. for 15 s, 56° C. for 20 s and 72° C. for 30 s, using a Step One Plus machine (Applied Biosystem). The relative mRNA expression was calculated accordingly to the 2\hat{ }(−ΔCt) method comparing the expression of different genes to that of GAPDH housekeeping. Primers were designed using the software PRIMER3 (http://frodo.wi.mit.edu/primer3/) using published data obtained from the NCBI database. The primer used for murine samples were conducted as follows (forward and reverse): Gapdh (for ctgagtatgtcgtggagtctac (SEQ ID: 1); rev gttggtggtgcaggatgcattg (SEQ ID: 2)), Tnf-α (for ccaccacgctcttctgtcta (SEQ ID: 3); rev agggtctgggccatagaact (SEQ ID: 4)), Il-6 (for cttcacaagtcggaggctta (SEQ ID: 5); rev ttctgcaagtgcatcatcgt (SEQ ID: 6)), Il-1β (for gctgaaagctctccacctca (SEQ ID: 7); rev aggccacaggtattttgtcg (SEQ ID: 8)), Il-10 (for cccagaaatcaaggagcatt (SEQ ID: 9); rev ctcttcacctgctccactgc (SEQ ID: 10)), Gpbar1 (for ggcctggaactctgttatcg (SEQ ID: 11); rev gtccctcttggctcttcctc (SEQ ID: 12)).

The primer used for human cell samples were conducted as follows (forward and reverse): GAPDH (for cagcctcaagatcatcagca (SEQ ID: 13); rev ggtcatgagtccttccacga (SEQ ID: 14)), TNFα (for agcccatgttgtagcaaacc (SEQ ID: 15); rev tgaggtacaggccctctgat (SEQ ID: 16)), IL-6 (for ccaggagaagattccaaagatg (SEQ ID: 17); rev ggaaggttcaggttgttttctg (SEQ ID: 18)), IL-113 (for gtggcaatgaggatgacttg (SEQ ID: 19); rev ggagattcgtagctggatgc (SEQ ID: 20)), IL-10 (for tgccttcagcagagtgaaga (SEQ ID 21); rev ctcagacaaggcttggcaac (SEQ ID 22)), GPBAR1 (for actgttgtccctcctctccct (SEQ ID 23); rev gacactgctttggctgcttg (SEQ ID 24)).

Statistical Analysis

The ANOVA followed by non parametric Mann-Whitney U test or a two-tailed unpaired Student t test were used for statistical comparisons (*P<0.05) using the Prism 6.0 software (GraphPad).

1. A compound of formula (I): 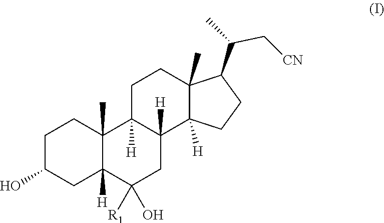
wherein:
R₁ is selected from the group consisting of linear or branched C₁-C₆ alkyl, linear or branched C₂-C₆ alkenyl, linear or branched C₂-C₆ alkynyl, benzyl and aryl
or pharmaceutically acceptable solvates or isomers thereof.

2. The compound of claim 1 wherein R₁ is selected from the group consisting of methyl, ethyl, n-propyl, isopropyl, 2-propenyl, vinyl, phenyl, 4-pentenyl and benzyl.

3. The compound of claim 1, wherein the compound is selected from the group consisting of:

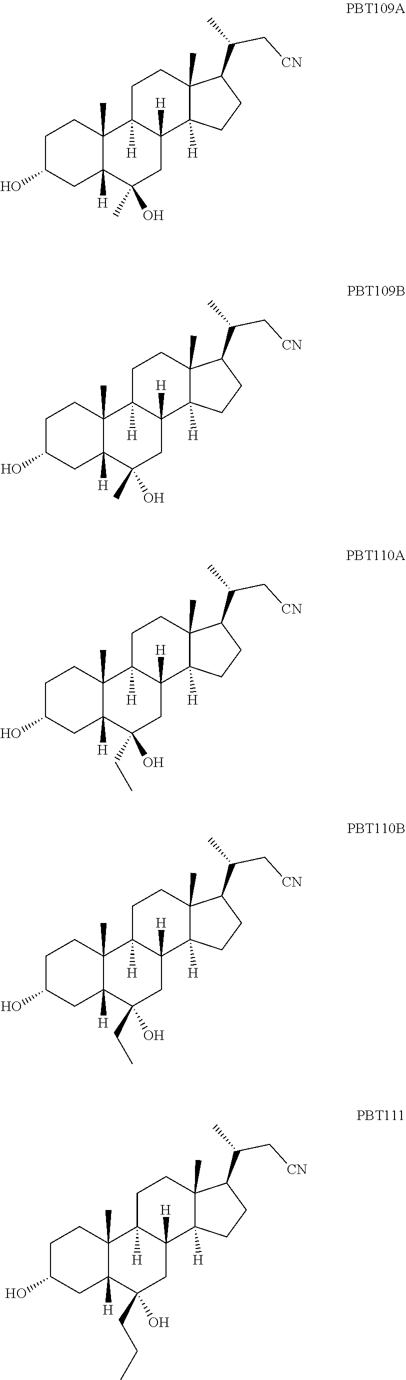

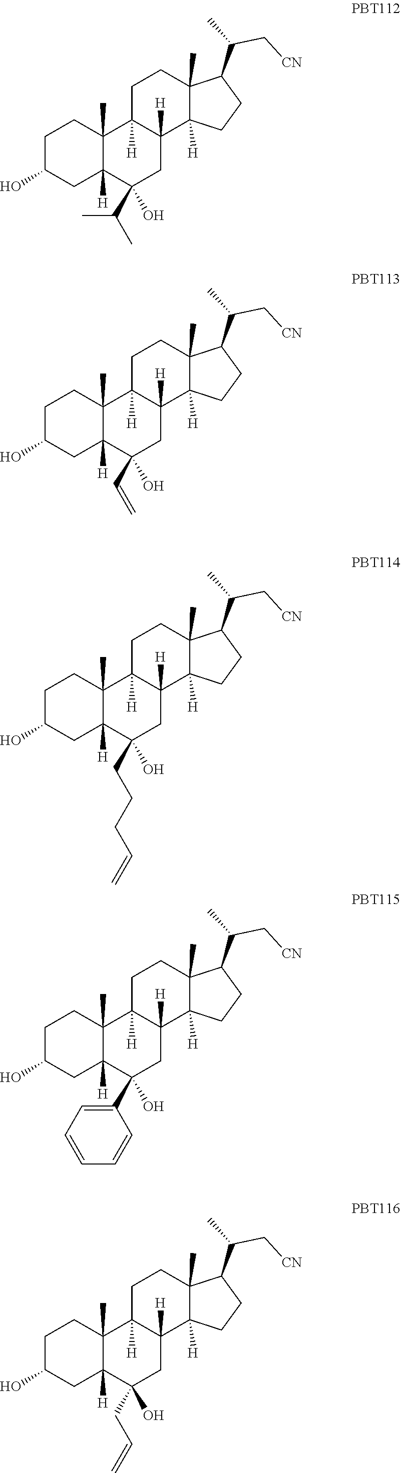

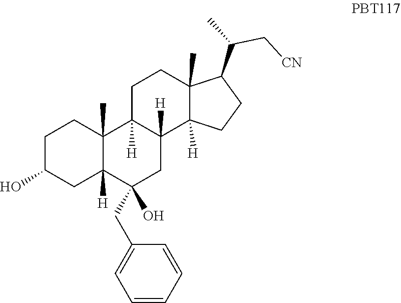

4. A pharmaceutical composition comprising a compound of claim 1 and at least one pharmaceutically acceptable excipient.

5. The compound of claim 1 for the use as a medicament.

6. A method for the prevention and/or treatment of gastrointestinal disorders, liver diseases, cardiovascular diseases, atherosclerosis, metabolic diseases, metabolic disorders, infectious diseases, cancer, renal disorders, inflammatory disorders, or neurological disorders, the method comprising administering a compound of claim 1 to a subject in need thereof.
